(19) United States
(12) Patent Application Publication
(10) Pub. No.: US 2007/0269849 A1
(43) Pub. Date: Nov. 22, 2007

(54) Air Sampling Method, Device and System for Microbiological Analysis

(76) Inventors: Pierre Bridenne, Forges-Les-Bains (FR); Pascale Coffinier, Lardy (FR)

Correspondence Address: YOUNG & THOMPSON, 745 SOUTH 23RD STREET, 2ND FLOOR, ARLINGTON, VA, 22202, US

(21) Appl. No.: 11/662,342

(22) PCT Filed: Oct. 6, 2005
(86) PCT No.: PCT/FR05/02455

§ 371 Date: Mar. 8, 2007

(30) Foreign Application Priority Data
Oct. 6, 2004 (FR) 0410514

(51) Int. Cl. C12Q 1/24; C12M 1/22

(52) U.S. Cl. 435/30; 435/288.3

(57) ABSTRACT

A method of using an air suction device is provided having a device equipped with a removable sampling head that is replaced between each sampling operation. Inside the head is a Petri dish containing a culture medium. Air is sucked through the head in order to impact on the culture medium. A device for implementing such a method includes a single-use head, in particular a head which must be destroyed in order to carry out the analysis. The device is especially suitable for use in a sampling system established in a fixed station in an industrial plant, clean room, work station, or operating area.

This invention relates to air-impacting sampling devices. Such devices are generally called bio-impactors. They are generally used for sampling micro-organisms in the air, this air being sucked in so that it impacts on a culture medium, for example agar contained in a Petri dish. Microbiological analysis of the air is carried out by observation of the microorganisms which develop in the culture medium.

The Petri dish is generally arranged inside a removable head. The head comprises an air inlet for the air, opposite the culture medium, and an air suction outlet at the rear of the dish. The air inlet generally comprises a screen comprising orifices for the passage of air, the number, size and arrangement of which orifices are chosen as a function of a sampling protocol.

The head must be completely sterile in order that any microorganisms originating from the head and not from the analyzed air, will not falsify the analysis. The head must therefore be sterilized between each sampling operation. The sterilization of the head by oven heating, as envisaged by current protocols, requires time, equipment and delicate handling on the part of an operator who is generally also responsible for the sampling operation. Very often this sterilization by oven heating is not carried out, the operator being content to clean the head with a disinfectant product. However, apart from the fact that the disinfection may therefore be incomplete, disinfectant can remain on the screen and be carried along by the air as far as the culture medium, which becomes at least partially sterile. The analysis thereof is thus falsified. In order to avoid this drawback, the operator is obliged to carry out several successive sampling operations in order to be able to verify them with each other.

The purpose of the invention is to propose a sampling method and/or device, making it possible to simplify the sampling protocols, whilst ensuring that the culture medium is not polluted by microorganisms foreign to the analyzed air or by a disinfectant product.

According to a first subject of the invention, such a method for sampling air with a view to microbiological analysis is such that:
an air suction device is used;
the device is equipped with a removable sampling head;
a Petri dish containing a culture medium is used, which dish is arranged inside the sampling head;
the air is sucked through the head in order to impact on said culture medium.

This method is characterized in that the head is changed between each sampling operation, i.e. the head is replaced with a new head.

The Petri dish can be provided fitted in the head and supplied thus, for example in a sterile package. The Petri dish can be supplied already with the culture medium, this medium being for example in the form of agar. In another method of implementation, the head can be supplied without the culture medium or even without the dish, which allows the operator to use his own culture medium, according to his choice.

The head is therefore provided disposable, produced from inexpensive materials and using inexpensive methods. In order to avoid voluntary or involuntary reuse of the head, once the sampling operation has been carried out, the head is preferably destroyed. The destruction can occur automatically depending on the needs of the analysis, for example on removing the dish in order to be able to observe the microbiological cultures therein.

The head is preferably obtained sterile during manufacture. For example, it can be manufactured in a sterile environment and/or sterilized, in particular by gamma radiation.

In order to maintain the sterility of the head, and particularly if it contains a culture medium, an air inlet and/or a suction outlet on the head can be closed, during its manufacture, with a cap which is removed before the sampling operation and/or assembling the head, respectively. This cap is preferably designed so as not to be repositionable, in order to limit the possibilities for reuse of the head. In another embodiment, the cap can be repositionable in order to be able to avoid pollution or drying-out of the culture medium, after the sampling operation. It can also be packaged in sterile manner, for example in a hermetically sealed sachet.

According to a second subject of the invention, a sampling device according to the invention comprises a single-use head to be used in a method as previously characterized. The head can comprise destructible means making it unsuitable for more than one sampling operation. If the Petri dish is fitted in the head, the destructible means comprise means for holding the dish in the head, said destructible means being designed to be destroyed during the removal of the dish with a view to analysis of the sample. These holding means can be provided for fitting of the dish in the head by clipping. They can be in the form of claws.

In another embodiment, the dish is cast in a single piece with the head. In this case too, the means for holding the part of the head forming a dish relative to the remainder of the head can be provided so as to be destructible. A cover forming an air inlet can be fitted on the head opposite the culture medium in the dish. Before it is put on, this cover in particular allows access to the dish for putting the culture medium in it and/or after its removal for observation of the cultures. The destructible means can comprise means for fixing the cover on the head.

In order to be able to use a device according to the invention with a bio-impactor of the prior art, adapting means can be provided for fixing the head onto the body of the bio-impactor.

Advantageously, a device according to the invention can comprise at least two heads, the first of which comprises a suction outlet suitable for being fixed onto the body of a bio-impactor, and a second head, the first and the second head comprising a complementary air inlet and suction outlet respectively in order to fit the suction outlet of the second head onto the complementary inlet of the first, for example by fitting together. The device can comprise at least two second heads, comprising a complementary inlet and outlet for fitting one in a cascading manner over the other. By way of example, the complementary inlets and outlets are substantially tapered, and are fitted one over the other by wedging one cone into the other.

The head can comprise means for adapting an isokinetic sampling probe to it. The device can be designed in order to be arranged in a flow of air virtually without disturbing said air flow. It can comprise means for improving its aerodynamism, and/or the head can itself be substantially aerodynamic in shape. This is particularly advantageous for carrying out sampling operations in a unidirectional flow, in particular in a laminar flow.

In particular in agri-food or pharmaceutical production lines, sampling operations are carried out regularly, at strategic points on the line. According to a third subject of the invention, a sampling system comprising a fixed pipeline and a suction pump can advantageously be provided. The pipeline is then arranged in order to link one or more locations provided for fixing there a device according to the invention with said pump. Thus, the sampling operations are carried out systematically at the same point, with the same position of the head and their results can be compared. The sampling conditions are therefore made reproducible. Moreover, it is sufficient for an operator to fix a sampling device according to the invention at each location provided, then to simultaneously carry out the sampling operations at these locations. Such a system advantageously comprises means for regulating the flow rate of air in the pipeline. The regulation can be independent for each location, in particular depending on a sampling protocol. Such a system can also be used at a work station, in a clean room or in an operating area. The sampling operations can be carried out continuously, over a given period or at regular intervals.

By the term air in this description is meant any gaseous mixture capable of being analyzed using a device according to the invention, or using a method according to the invention.

Other features and advantages of the invention will also be apparent from the description hereafter, relating to non-limitative examples.

In the attached drawings:

FIG. 1 is a cross-section of a first part of a sampling head in a device according to the invention;

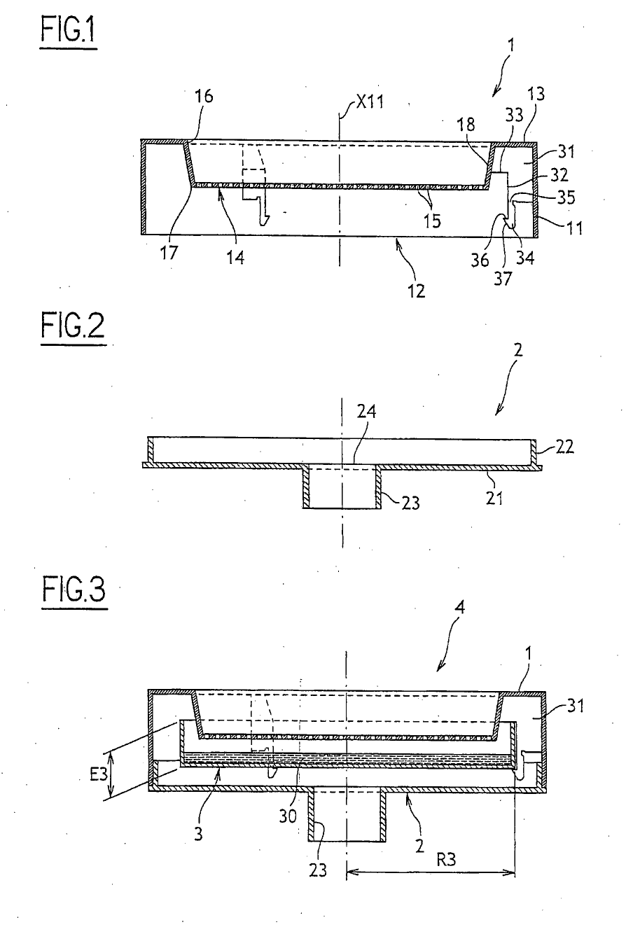

FIG. 2 is a cross-section of a second part of the head for fitting the part of FIG. 1 thereon;

FIG. 3 is a cross-section of the assembled parts of FIGS. 1 and 2, a Petri dish being fitted inside the head;

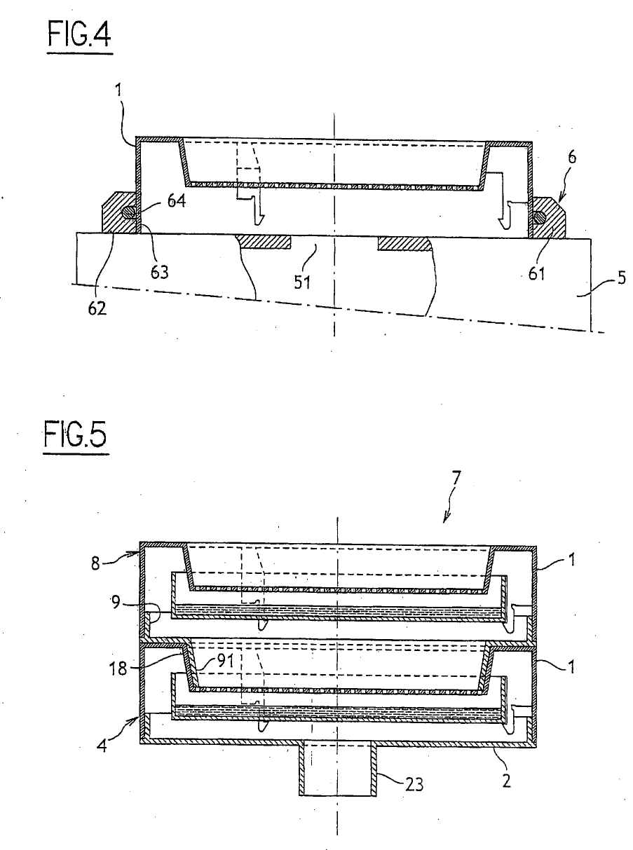

FIG. 4 is a cross-section of a head comprising only the first part and fitted onto the body of a bio-impactor of the prior art, using adapting means; and,

FIG. 5 is a cross-section of a sampling device formed by two heads fitted in a cascading manner one over the other.

FIG. 1 represents a first part 1 of a sampling head for a sampling device according to the invention. This first part can be completed by a second part 2, as illustrated in FIG. 3 or used alone, as illustrated in FIG. 4.

Each of the two parts 1, 2 is moulded from a single piece which is made of a plastic material.

The first part comprises an outer wall 11 in the form of a cylinder with an axis X11. The cylinder is open at a first end 12. This end forms a suction outlet 12 for the first part 1. The first part 1 comprises, at a second end of the cylinder, axially opposite to the first end, an annular wall 13 extending radially towards the axis X11 of the cylinder 11, between the outer wall 11 and a circular inner edge 16 of the annular wall 13.

A screen 14 serves as an inlet for the air to be sampled. This screen is in the shape of a disc perforated with orifices 15, distributed substantially over the whole disc, for the passage of air. The screen is arranged between the two ends of the cylinder 11. The outer edge 17 of the screen 14 is connected to the inner edge 16 of the annular wall, by a tapered connecting wall 18. The tapered wall cone 18 widens from the outer edge 17 of the screen towards the inner edge 17 of the annular wall 13.

The first part is provided for fitting therein a substantially cylindrical Petri dish 3 containing agar 30 forming a culture medium for micro-organisms.

The first part 1 also comprises means for holding a Petri dish 3. These holding means comprise three flat ribs 31 extending radially from the outer wall 11, and parallel to the axis X11 from the annular wall 13. The holding means also comprise three claws 34, each claw extending from a respective rib 31, parallel to the axis X11.

The three ribs and associated claws are substantially identical with each other. They are distributed regularly around the axis X11. The ribs form, each with a claw, an angle bracket 32, 33 a longitudinal edge 32 of which, i.e. parallel to the axis X11, has a length L32 approximately equal to the thickness E3 of the Petri dish 3 provided to be held there. The radial distance between the longitudinal edge 32 and the axis X11 is substantially equal to the radius R3 of the Petri dish 3. A radial edge 33 of the angle bracket extends between the tapered wall 18 and one end of the longitudinal edge closest to the annular wall 13.

Each claw 34 is connected to its rib by a connecting zone 35 with a narrowed section. The claw also comprises a support edge 36, extending radially from the other end of the longitudinal edge 32, opposite the radial edge 33. A bevelled edge 37 extends it from the support edge 36 away from both the axis X11 and the annular wall 13.

During the fitting of the dish 3 in the first part 1 of the head, the dish is supported against the bevelled edges, pushing back the claws which bend is a substantially elastic fashion at the level of the connecting zone 35. When the dish buts up against the radial edges 33, the claws bend back in such a manner that the dish 3 is held axially, gripped between the radial edges 33 and the support edges 36. Moreover, the dish is centred radially between the longitudinal edges 32.

The screen is positioned in the first part in such a manner that, when the dish is fitted, the screen is inside the dish 3, opposite the surface of the agar intended to receive the micro-organisms.

During the removal of the dish it is necessary to break the claws in order to release the dish. The first part is then unusable. This first part must therefore be systematically changed in order to carry out a new sampling operation. It is this first part, extending from upstream to downstream of the Petri dish relative to the flow of air during the sampling operation, which is likely to introduce contamination onto the agar. Its replacement with a new first sterile part makes it possible to ensure that such contamination does not occur.

FIG. 2 illustrates a second complementary part for the head 4 illustrated in FIG. 3. This second part forms a suction outlet 2 for the head 4.

This second part 2 comprises an annular disc 21, with a hole drilled through its centre. A fitting cylinder 22, complementary to the outer wall 11 of the first part 1 extends from one side of the annular disc 21. This cylinder 22 is provided in order to be inserted inside the outer wall 11, by fitting together and wedging, in substantially airtight manner.

A fixing cylinder 23, extends from the other side of the disc in order to be fixed in substantially airtight manner on a complementary intake, not shown, of a suitable bio-impactor.

FIG. 4 illustrates the fitting of a head 1, constituted only by the first part illustrated in FIG. 1, on a bio-impactor 5 which is not suitable for the use of a head 4, as illustrated in FIG. 3. This is generally the case for bio-impactors of the prior art.

A device according to the invention then comprises, besides the head 1, adapting means 6 for fixing the head 1 on the bio-impactor 5. In the example illustrated, the adapting means comprise a ring 61. This ring 61 comprises a bearing surface 62, fixed in substantially airtight manner against the bio-impactor 5, centred around a suction port 51 of the bio-impactor.

The ring comprises a cylindrical inner surface 63 the diameter of which is slightly greater than the outer diameter of the outer wall 11 of the head 1. An O-ring 64, fitted in the ring 6 is flush with the cylindrical surface 63, in such a manner that when the head 1 is fitted on the bio-impactor, the joint forms an airtight seal against the outer wall 11 of the head, and elastically holds the head 1 on the bio-impactor 5.

The device 7 illustrated in FIG. 5 comprises two heads 4, 8. A first head 4 is identical to that described with reference to FIG. 3. A second head 8 comprises a first part 1 identical to that of the first head 4. The second part 9 of the second head 8 differs from the second part 2 of the first head 4, in that the fixing cylinder 23 is replaced by a tapered wall 91 complementary to the tapered wall 18 of the first part 1. Thus, the tapered walls 18, 91, can be fitted one inside the other, allowing the assembling in a cascading manner of the two heads 4, 8.

Such a device 7 is particularly suitable for collecting in the first head micro-organisms which, on account of their nature, size, or air flow conditions in the device, have not been able to be collected in the second head 8. Other second heads can be fitted in a cascading manner on the second head 8 described previously, in order to refine the sampling operation.

In order to guarantee the sterility of the zones of the path of the air in the head, a first cap can be provided, attached in a removable manner onto the annular wall 11 of the first part 1, and a second cap can be attached in a removable manner so as to block off the first end 12 of the cylinder forming the outer wall 11 of this first part 1.

If the head is supplied assembled as illustrated in FIG. 3, the second cap can be attached so as to block off the fixing cylinder 23.

Of course, the invention is not limited to the examples which have just been described and numerous changes can be made to these examples without exceeding the scope of the invention.

In particular, the shapes and number of the different pieces described can be different. For example, the ribs and the claws can be of a number different from three.

For a sampling system according to the invention set up in a fixed station in an industrial plant, a location for fixing a head there can be similar to a second part as described with reference to FIG. 2. The cylinder 23 can then be replaced by a flexible or rigid pipe, making it possible to link said location, from the sampling point, to an air suction pump.

The device can comprise means for verifying the flow rate of air in the head. These means can make it possible to adjust this flow rate, for example in order to compensate for manufacturing tolerances or operator-fitting tolerances.

In order to maintain a suitable distance between the surface of the culture medium and the screen, dimensions of the head can vary and/or be adjustable. Thus, the head can comprise means of adjustment, for example of the position of the claws and/or of the position of the screen. This suitable distance can be a function of the type of Petri dish and/or of a sought type of micro-organisms. The adjustment is preferably carried out in the factory.

1. Method for carrying out an air sampling operation with a view to microbiological analysis such that:
an air suction device is used;
the device is equipped with a removable sampling head (4);
a Petri dish (3) containing a culture medium (30) is used, arranged inside the sampling head;
the air is sucked through the head in order to impact on said culture medium;
characterized in that once the sampling operation has been carried out, the head is destroyed and in that the head is replaced between each sampling operation.

2. Method according to claim 1, characterized in that the Petri dish is provided fitted in the head.

3. Method according to claim 2, characterized in that removal of the Petri dish causes the destruction of the head.

4. Method according to claim 3, characterized in that means (34) for holding the Petri dish in the head must be broken in order to remove said dish from said head.

5. Method according to claim 1, wherein prior to the fitting of the head, a cap is removed from a suction outlet of the head.

6. Method according to claim 1, wherein prior to the sampling operation, a cap is removed from an air inlet of the head.

7. Sampling device comprising a single-use head to be used in a method according to claim 1, said device comprising means for fixing the head onto an air-suction device, and said head comprising at least one air inlet forming a screen.

8. Device according to claim 7, characterized in that it comprises a cap on the air inlet (13, 15, 18) and/or a cap on a suction outlet (12, 23), said cap being removable.

9. Device according to claim 8, characterized in that the cap is not repositionable.

10. Device according to claim 7, wherein the head comprises destructible means making it unfit for more than one sampling operation.

11. Device according to claim 10, characterized in that the destructible means comprise means (31) for holding a Petri dish (3) in the head, said destructible means being designed to be destroyed during the removal of the dish.

12. Device according to claim 7, wherein a Petri dish is formed in a single piece with the head.

13. Device according to claim 11, characterized in that the holding means comprise means (37) of fitting the dish into the head by clipping.

14. Device according to claim 11, wherein the holding means comprise a form of claw.

15. Device according to claim 7, wherein the sampling device comprises a cover forming an air inlet arranged to be fitted onto the head opposite a culture medium in the Petri dish.

16. Device according to claim 15, characterized in that the destructible means comprise means for fixing the cover.

17. Device according to claim 7, comprising adapting means for fixing the head onto the body of a bio-impactor of the prior art.

18. Device according to claim 7, comprising at least two heads a first of which comprises a suction outlet suitable for being fixed onto the body of a bio-impactor, and a second head, the first head and the second comprising respectively a complementary air inlet and suction outlet for fitting the suction outlet of the second head onto the complementary inlet of the first head.

19. Device according to claim 18, comprising at least two second heads comprising a complementary inlet and outlet for fitting one over the other.

20. Device according to claim 18, wherein the complementary inlets and outlets are substantially tapered.

21. Device according to 7, wherein the head comprises means for adapting an isokinetic sampling probe to it.

22. Device according to claim 7, suitable for being arranged in a flow of air, substantially without disturbing said flow of air.

23. Device according to claim 7, comprising two parts, a first part comprising an inlet for the air, the second part comprising a suction outlet, the two parts being able to be fitted one over the other in substantially airtight manner.

24. Device according to claim 23, characterized in that the first part comprises means (31) for holding the dish.

25. Device according to claim 7, wherein the screen is opposite a surface of a culture medium, said device comprising means for adjusting a distance between said screen and said surface of the culture medium.

26. Air-sampling system, comprising a fixed pipeline and a suction pump, said
